(19) United States
(12) Patent Application Publication
(10) Pub. No.: US 2025/0299832 A1
(43) Pub. Date: Sep. 25, 2025

(54) CARDIAC BUNDLE BRANCH BLOCK MODELING

(71) Applicant: Siemens Medical Solutions USA, Inc., Malvern, PA (US)

(72) Inventors: Viorel Miihalef, North Brunswick, NJ (US); Tiziano Passerini, Plainsboro, NJ (US); Puneet Sharma, Princeton Junction, NJ (US)

(21) Appl. No.: 18/609,190

(22) Filed: Mar. 19, 2024

(51) Int. Cl. G16H 50/50; A61B 5/319

(52) U.S. Cl. CPC G16H 50/50; A61B 5/319

(57) ABSTRACT

Systems and methods for optimizing an electro-anatomical model of an anatomical object of a patient are provided. 1) one or more input medical images of an anatomical object of a patient and 2) electrophysiological data associated with the anatomical object are received. An electro-anatomical model of the anatomical object is generated based on the one or more input medical images and the electrophysiological data. The electro-anatomical model is optimized based on one or more electrical conduction parameters for electrical pathways in the electro-anatomical model. The one or more electrical conduction parameters are continuous variables defined between a value representing no electrical conduction in the electrical pathways and a value representing full electrical conduction in the electrical pathways. The optimized electro-anatomical model is output.

### STATEMENT REGARDING FEDERALLY SPONSORED RESEARCH OR DEVELOPMENT.

This invention was made with government support under R01 HL159945 awarded by the National Institutes of Health. The government has certain rights in the invention.

### TECHNICAL FIELD

The present invention relates generally to an electro-anatomical model of an anatomical object of a patient, and in particular to improved cardiac bundle branch block modeling.

### BACKGROUND

Bundle branches (or Tawara branches) are offshoots of the bundle of His in the ventricle of the heart. Bundle branches play an important role in the electrical conduction system of the heart by transmitting cardiac action potentials from the bundle of His to the Purkinje fibers. The bundle of His comprises two bundle branches: the left bundle branch and the right bundle branch. The left bundle branch further divides into the left anterior fascicle and the left posterior fascicle. Each branch or fascicle may further comprise additional offshoots.

A bundle branch or their fascicles may become injured due to, for example, an underlying heart disease, myocardial infarction, or cardiac surgery, resulting in blocked electrical pathways, a condition known as bundle branch block. Bundle branch block causes the left and right sides of the heart to beat out of sync and pathologically lengthens the total depolarization duration of the heart. In the current clinical workflow, bundle branch block is typically treated with CRT (cardiac resynchronization therapy) by implanting a pacemaker system in the chest of a patient to coordinate the contraction of the heart. However, approximately one-third of CRT treatments are not successful long term. Recently, computational models have been proposed for optimizing CRT. However, such conventional computational models are not sufficiently refined and are limited in their ability to be personalized.

### BRIEF SUMMARY OF THE INVENTION

In accordance with one or more embodiments, systems and methods for optimizing an electro-anatomical model of an anatomical object of a patient are provided. 1) one or more input medical images of an anatomical object of a patient and 2) electrophysiological data associated with the anatomical object are received. An electro-anatomical model of the anatomical object is generated based on the one or more input medical images and the electrophysiological data. The electro-anatomical model is optimized based on one or more electrical conduction parameters for electrical pathways in the electro-anatomical model. The one or more electrical conduction parameters are continuous variables defined between a value representing no electrical conduction in the electrical pathways and a value representing full electrical conduction in the electrical pathways. The optimized electro-anatomical model is output.

In one embodiment, the electro-anatomical model is optimized by activating a proportion of the electrical pathways in the electro-anatomical model determined based on the one or more electrical conduction parameters. In one embodiment, the electro-anatomical model is optimized using conduction velocity parameters determined by multiplying a maximal conduction velocity of a fascicle by the one or more electrical conduction parameters. In one embodiment, the electro-anatomical model is optimized by delaying electrical activation in the electrical pathways based on the one or more electrical conduction parameters.

In one embodiment, a medical procedure on the anatomical object is simulated using the optimized electro-anatomical model. In one embodiment, the anatomical object is a heart of the patient and different types of cardiac resynchronization therapy are simulated for determining an optimal treatment for bundle branch block of the electrical pathways. The different types of cardiac resynchronization therapy may comprise left ventricle pacing, bi-ventricular pacing, His bundle pacing, and Left bundle branch pacing.

In one embodiment, the anatomical object comprises a heart of the patient, the value representing no electrical conduction in the electrical pathways corresponds to complete conduction block, and the value representing full electrical conduction in the electrical pathways corresponds to intact Purkinje activation.

In one embodiment, the anatomical object comprises a heart of the patient and the electrical pathways comprise at least one of a bundle of His, a right bundle branch, a left branch, Purkinje fibers, or any sub-branch thereof.

These and other advantages of the invention will be apparent to those of ordinary skill in the art by reference to the following detailed description and the accompanying drawings.

### BRIEF DESCRIPTION OF THE DRAWINGS

FIG. 1 shows a workflow for optimizing a cardiac electro-anatomical model of a heart of a patient, in accordance with one or more embodiments;

FIG. 2 shows a method for optimizing an electro-anatomical model of an anatomical object of a patient, in accordance with one or more embodiments; and

FIG. 3 shows a high-level block diagram of a computer that may be used to implement one or more embodiments.

### DETAILED DESCRIPTION

The present invention generally relates to methods and systems for improved cardiac bundle branch block modeling. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Conventional cardiac models are optimized by representing electrical conduction parameters of bundle blocks in a binary one/off manner. Embodiments described herein provide for optimization of a cardiac model by defining electrical conduction parameters as continuous variables between, for example, 0 (representing complete conduction block) and 1 (representing intact Purkinje activation). The electrical conduction parameters may be defined for the left or right bundle blocks, for their fascicles, or for any other point on the bundle blocks/fascicles by defining a separate electrical conduction parameter for each. Advantageously, optimized cardiac models, optimized in accordance with embodiments described herein, may be used for more accurately performing virtual CRT (cardiac resynchronization therapy) as compared with conventional cardiac models, to thereby determine the specifics of the best CRT activation modality for optimizing activation timing and location.

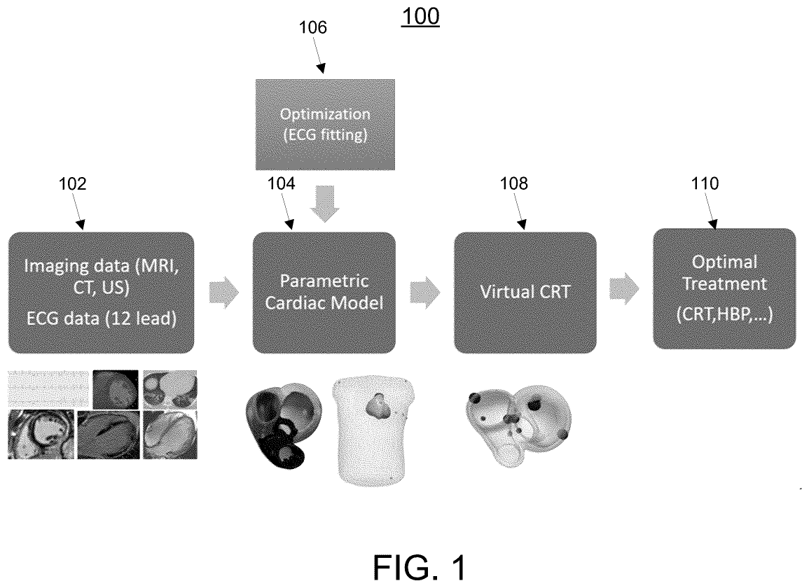

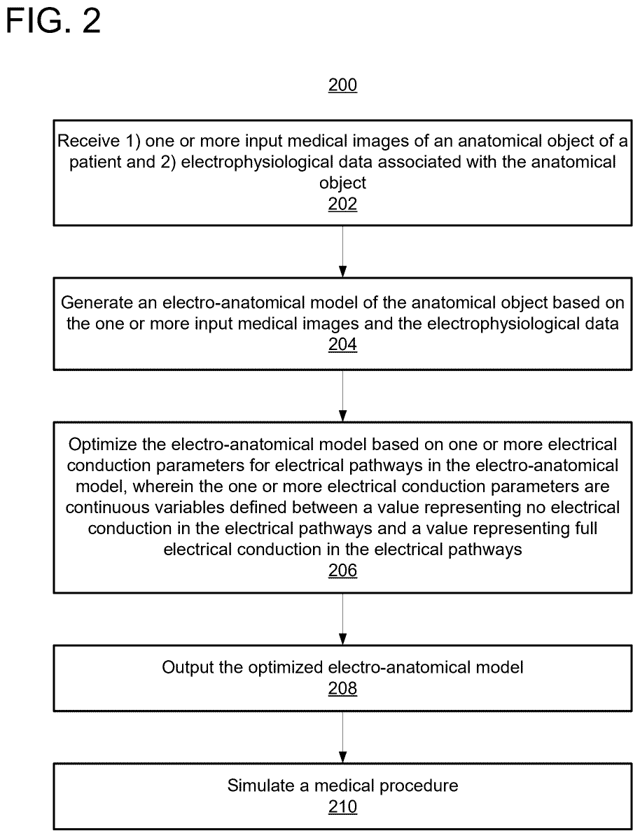

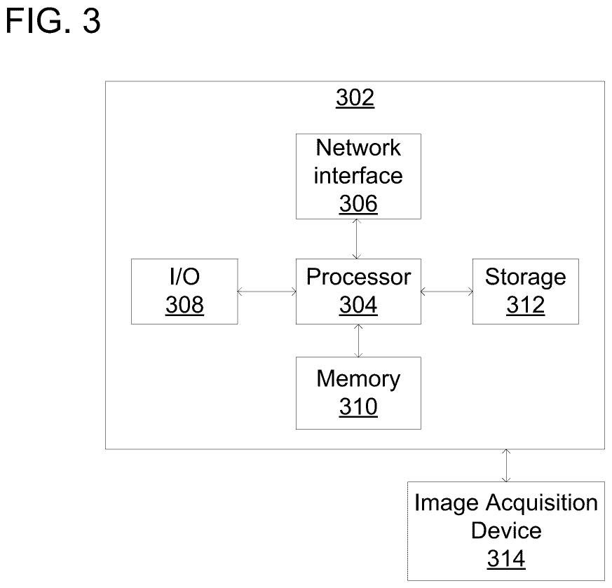

FIG. 1 shows a workflow 100 for optimizing a cardiac electro-anatomical model of a heart of a patient, in accordance with one or more embodiments. FIG. 2 shows a method 200 for optimizing an electro-anatomical model of an anatomical object of a patient, in accordance with one or more embodiments. The steps of method 200 may be performed by one or more suitable computing devices, such as, e.g., computer 302 of FIG. 3. FIG. 1 and FIG. 2 will be described together.

At step 202 of FIG. 2, 1) one or more input medical images of an anatomical object of a patient and 2) electrophysiological data associated with the anatomical object are received. In one embodiment, the anatomical object is the heart of the patient. For example, as shown in workflow 100 of FIG. 1, the one or more input medical images and the electrophysiological data is patient data 102 comprising imaging data and ECG (electrocardiogram) data of the heart of the patient. However, the anatomical object may be any other suitable anatomical object or objects of interest of the patient, such as, e.g., the brain, muscles, nerves, the pancreas, the gastrointestinal tract, the retina, etc.

The one or more input medical images may be of any suitable modality, such as, e.g., MRI (magnetic resonance imaging), CT (computed tomography), US (ultrasound), x-ray, or any other medical imaging modality or combinations of medical imaging modalities. The one or more input medical images may be 2D (two dimensional) images and/or 3D (three dimensional) volumes, and may comprise a single input medical image or a plurality of input medical images.

In one embodiment, where the anatomical object is the heart of the patient, the electrophysiological data may comprise ECG data of the torso (e.g., acquired either as a standard 12-lead ECG based on 9 electrode locations, or at an extended set of torso locations, for example the 252 electrode Cardiolnsight vest) or electro-anatomical mapping data of the heart acquired by measuring the electrical potential at one or more anatomical points of the heart via a catheter. Examples of electro-anatomical mapping data of the heart may include local activation time and potentials (e.g., for a cardiac chamber or chambers in relation to a timing of a reference electrogram), unipolar or bipolar voltage, fractionation electrograms, and propagation (e.g., showing spread of activation wave front throughout a cardiac cycle, or showing direction vectors of the wave propagation). However, the electrophysiological data may be any other suitable electrophysiological data of the patient, such as, e.g., EEG (electroencephalogram) data, EMG (electromyogram) data, NCS (nerve conduction studies) data, ERG (electroretinogram) data, EGG (electrogastrogram), ECoG (electrocorticography) data, etc.

The one or more input medical images and/or the electrophysiological data may be received, for example, by directly receiving the one or more input medical images from an image acquisition device (e.g., image acquisition device 314 of FIG. 3) as the medical images are acquired, by loading previously acquired medical images and/or electrophysiological data from a storage or memory of a computer system (e.g., storage 312 or memory 310 of computer 302 of FIG. 3), or by receiving medical images and/or electrophysiological data from a remote computer system (e.g., computer 302 of FIG. 3). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 204 of FIG. 2, an electro-anatomical model of the anatomical object is generated based on the one or more input medical images and the electrophysiological data. In one example, as shown in workflow 100 of FIG. 1, the electro-anatomical model is generated as parametric cardiac model 104 generated from patient data 102. The electro-anatomical model is a computational model for simulating the electrophysiology of the anatomical object. In one embodiment, the electro-anatomical model is generated by first generating an anatomical model of the anatomical object and applying an electrophysiology model to the anatomical model. However, the electro-anatomical model may be generated according to any other (e.g., well known) approach.

To generate the anatomical model of the anatomical object, the anatomical object first is segmented from the one or more input medical images (e.g., using a machine learning based segmentation model) and a surface mesh representing the geometry of the anatomical object is generated (e.g., using a surface reconstruction algorithm, such as, e.g., marching cubes or level set methods). The surface mesh is then converted into a volumetric tetrahedral mesh using, e.g., a mesh generation algorithm to fill the volume enclosed by the surface mesh with tetrahedral elements while satisfying certain criteria (e.g., element quality, mesh density). The tetrahedral mesh represents the anatomical model of the anatomical object. The anatomical object may be personalized to the patient to best fit a meaningful metric to the one or more input medical images.

Once the anatomical model is generated, an electrophysiology model is applied to the anatomical model to generate the electro-anatomical model. The electrophysiology model models the propagation of electrical conduction over electrical pathways of the anatomical object. For example, where the anatomical object is the heart of the patient, the electrophysiology model may model the propagation of electrical signals in the heart during each cardiac cycle, as well as propagation to the torso. The electrophysiology model may include equations that govern, for example, the generation and propagation of action potentials along the electrical pathways, as well as parameters that characterize tissue conductivity, refractoriness, and other electrophysiological properties.

In one embodiment, the electrophysiology model comprises one or more of the following: 1) a natural electrical signal activation system, modeling the positions and delays for the natural (intrinsic) cardiac depolarization, 2) an external electrical signal activation system, modeling the positions and delays for a device (extrinsic) cardiac depolarization, 3) electrophysiology constitutive values, such as, e.g., conductivity (or diffusivity) values for the myocardium, scar, and the left and right fast conduction systems, and 4) an electrophysiology propagation engine, such as, e.g., eikonal, mono, or bidomain reaction diffusion system.

At step 206 of FIG. 2, the electro-anatomical model is optimized based on one or more electrical conduction parameters for electrical pathways in the electro-anatomical model. The one or more electrical conduction parameters are continuous variables defined between a value representing no electrical conduction in the electrical pathways and a value representing full electrical conduction in the electrical pathways. The one or more electrical conduction parameters may be defined as continuous variables, for example, by a user. In one example, as shown in workflow 100 of FIG. 1, parametric cardiac model 104 is optimized at block 106 by, e.g., ECG fitting.

In one embodiment, where the anatomical object is the heart, the electrical pathways may be bundle branches of the heart, such as, e.g., the bundle of His, the right bundle branch, the left branch, or Purkinje fibers. An electrical conduction parameter may be defined for one or more of the bundle branches. Accordingly, the one or more electrical conduction parameters may define electrical conduction in, e.g., the bundle of His, the right bundle branch, the left branch, Purkinje fibers, and/or any sub-branch thereof. However, the electrical pathways may be any other electrical pathway of the anatomical object, such as, e.g., cortical pathways, white matter tracts, and subcortical pathways of the brain, motor neuron pathways and motor end plates of the muscles, and peripheral nerve pathways and autonomic nervous system pathways of the nerves.

The one or more electrical conduction parameters are defined as continuous variables, which can have any value between the value representing no electrical conduction in the electrical pathways and the value representing full electrical conduction in the electrical pathways. For example, the one or more electrical conduction parameters may be defined to have a value between 0 (representing no electrical conduction in the electrical pathways) and 1 (representing full electrical conduction in the electrical pathways). Where the anatomical object is the heart, the value representing no electrical conduction in the electrical pathways may correspond to complete conduction block and the value representing full electrical conduction in the electrical pathways may correspond to intact Purkinje activation.

In one embodiment, where the electrical conduction parameter is set to 0 (representing no electrical conduction in the electrical pathways) the entire electrical pathway is deactivated in the electro-anatomical model and where the electrical conduction parameter is set to 1 (representing full electrical conduction in the electrical pathways) the entire electrical pathway is activated in the electro-anatomical model. In another embodiment, where the electrical conduction parameter is set to a fractional value f between 0 and 1, the electrical pathways are partially activated. For example, the electro-anatomical model may be optimized by activating a proportion of the electrical pathways (based on fractional value f) (e.g., from the AV (atrioventricular) node to the activation root points or a total length of fascicles for the AV node to the downstream electrophysiological territories, if defined). In another example, the electro-anatomical model may be optimized using conduction velocity parameters determined by multiplying a maximal conduction velocity of a fascicle by the fraction f. In a further example, the electro-anatomical model may be optimized by delaying electrical activation in the electrical pathways based on the fraction f. The delay may be defined as: delay˜k(1−f)^{p}, where k is a proportionality parameter and p is a power parameter.

The electro-anatomical model is optimized using the one or more electrical conduction parameters, along with other optimization parameters (e.g., conduction velocities and/or delays, activation positions, etc.). The electro-anatomical model may be optimized using a gradient free optimizer, such as, e.g., BOBYQA (Bound Optimization BY Quadratic Approximation).

At step 208 of FIG. 2, the optimized electro-anatomical model is output. The optimized electro-anatomical model may also be output by, for example, storing the optimized electro-anatomical model on a memory or storage of a computer system (e.g., memory 310 or storage 312 of computer 302 of FIG. 3) or by transmitting optimized electro-anatomical model to a remote computer system (e.g., computer 302 of FIG. 3).

At step 210 of FIG. 2, one or more medical procedures on the anatomical object are simulated using the optimized electro-anatomical model. In one embodiment, where the anatomical object is the heart, the one or more simulated medical procedures are different types of CRT to determine an optimal treatment. In one example, as shown in workflow 100 of FIG. 1, virtual CRTs 108 are performed based on parametric cardiac model 104 as optimized at block 106 to determine optimal treatments 110. The simulated CRTs may be, for example, 1) CRT-LVP (left ventricle pacing) with a pacing lead placed on the left ventricle epicardium, 2) CRT-BVP (bi-ventricular pacing) with pacing leads placed on the left ventricular epicardium and in the right ventricular apex, 3) HBP (His bundle pacing) with a lead placed mid-septal basal, and/or 4) LBBP (left bundle branch pacing) with a lead placed mid-septal. An optimal modality of CRT may be output based on results of the simulations.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1 or 2. Certain steps or functions of the methods and workflows described herein, including one or more of the steps or functions of FIG. 1 or 2, may be performed by a server or by another processor in a network-based cloud-computing system. Certain steps or functions of the methods and workflows described herein, including one or more of the steps of FIG. 1 or 2, may be performed by a client computer in a network-based cloud computing system. The steps or functions of the methods and workflows described herein, including one or more of the steps of FIG. 1 or 2, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1 or 2, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 302 that may be used to implement systems, apparatuses, and methods described herein is depicted in FIG. 3. Computer 302 includes a processor 304 operatively coupled to a data storage device 312 and a memory 310. Processor 304 controls the overall operation of computer 302 by executing computer program instructions that define such operations. The computer program instructions may be stored in data storage device 312, or other computer readable medium, and loaded into memory 310 when execution of the computer program instructions is desired. Thus, the method and workflow steps or functions of FIG. 1 or 2 can be defined by the computer program instructions stored in memory 310 and/or data storage device 312 and controlled by processor 304 executing the computer program instructions. For example, the computer program instructions can be implemented as computer executable code programmed by one skilled in the art to perform the method and workflow steps or functions of FIG. 1 or 2. Accordingly, by executing the computer program instructions, the processor 304 executes the method and workflow steps or functions of FIG. 1 or 2. Computer 302 may also include one or more network interfaces 306 for communicating with other devices via a network. Computer 302 may also include one or more input/output devices 308 that enable user interaction with computer 302 (e.g., display, keyboard, mouse, speakers, buttons, etc.).

Processor 304 may include both general and special purpose microprocessors, and may be the sole processor or one of multiple processors of computer 302. Processor 304 may include one or more central processing units (CPUs), for example. Processor 304, data storage device 312, and/or memory 310 may include, be supplemented by, or incorporated in, one or more application-specific integrated circuits (ASICs) and/or one or more field programmable gate arrays (FPGAs).

Data storage device 312 and memory 310 each include a tangible non-transitory computer readable storage medium. Data storage device 312, and memory 310, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 308 may include peripherals, such as a printer, scanner, display screen, etc. For example, input/output devices 308 may include a display device such as a cathode ray tube (CRT) or liquid crystal display (LCD) monitor for displaying information to the user, a keyboard, and a pointing device such as a mouse or a trackball by which the user can provide input to computer 302.

An image acquisition device 314 can be connected to the computer 302 to input image data (e.g., medical images) to the computer 302. It is possible to implement the image acquisition device 314 and the computer 302 as one device. It is also possible that the image acquisition device 314 and the computer 302 communicate wirelessly through a network. In a possible embodiment, the computer 302 can be located remotely with respect to the image acquisition device 314.

Any or all of the systems, apparatuses, and methods discussed herein may be implemented using one or more computers such as computer 302.

One skilled in the art will recognize that an implementation of an actual computer or computer system may have other structures and may contain other components as well, and that FIG. 3 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving 1) one or more input medical images of an anatomical object of a patient and 2) electrophysiological data associated with the anatomical object; generating an electro-anatomical model of the anatomical object based on the one or more input medical images and the electrophysiological data; optimizing the electro-anatomical model based on one or more electrical conduction parameters for electrical pathways in the electro-anatomical model, wherein the one or more electrical conduction parameters are continuous variables defined between a value representing no electrical conduction in the electrical pathways and a value representing full electrical conduction in the electrical pathways; and outputting the optimized electro-anatomical model.

Illustrative embodiment 2. The computer-implemented method according to illustrative embodiment 1, wherein optimizing the electro-anatomical model based on one or more electrical conduction parameters for electrical pathways in the electro-anatomical model comprises: optimizing the electro-anatomical model by activating a proportion of the electrical pathways in the electro-anatomical model determined based on the one or more electrical conduction parameters.

Illustrative embodiment 3. The computer-implemented method according to any one of illustrative embodiments 1-2, wherein optimizing the electro-anatomical model based on one or more electrical conduction parameters for electrical pathways in the electro-anatomical model comprises: optimizing the electro-anatomical model using conduction velocity parameters determined by multiplying a maximal conduction velocity of a fascicle by the one or more electrical conduction parameters.

Illustrative embodiment 4. The computer-implemented method according to any one of illustrative embodiments 1-3, wherein optimizing the electro-anatomical model based on one or more electrical conduction parameters for electrical pathways in the electro-anatomical model comprises: optimizing the electro-anatomical model by delaying electrical activation in the electrical pathways based on the one or more electrical conduction parameters.

Illustrative embodiment 5. The computer-implemented according to any one of illustrative embodiments 1-4, further comprising: simulating a medical procedure on the anatomical object using the optimized electro-anatomical model.

Illustrative embodiment 6. The computer-implemented method according to illustrative embodiment 5, wherein the anatomical object is a heart of the patient and simulating a medical procedure on the anatomical object using the optimized electro-anatomical model comprises: simulating different types of cardiac resynchronization therapy for determining an optimal treatment for bundle branch block of the electrical pathways.

Illustrative embodiment 7. The computer-implemented method according to illustrative embodiment 6, wherein the different types of cardiac resynchronization therapy comprise left ventricle pacing, bi-ventricular pacing, His bundle pacing, and Left bundle branch pacing.

Illustrative embodiment 8. The computer-implemented method according to any one of illustrative embodiments 1-7, wherein the anatomical object comprises a heart of the patient, the value representing no electrical conduction in the electrical pathways corresponds to complete conduction block, and the value representing full electrical conduction in the electrical pathways corresponds to intact Purkinje activation.

Illustrative embodiment 9. The computer-implemented method according to any one of illustrative embodiments 1-8, wherein the anatomical object comprises a heart of the patient and the electrical pathways comprise at least one of a bundle of His, a right bundle branch, a left branch, Purkinje fibers, or any sub-branch thereof.

Illustrative embodiment 10. An apparatus comprising: means for receiving 1) one or more input medical images of an anatomical object of a patient and 2) electrophysiological data associated with the anatomical object; means for generating an electro-anatomical model of the anatomical object based on the one or more input medical images and the electrophysiological data; means for optimizing the electro-anatomical model based on one or more electrical conduction parameters for electrical pathways in the electro-anatomical model, wherein the one or more electrical conduction parameters are continuous variables defined between a value representing no electrical conduction in the electrical pathways and a value representing full electrical conduction in the electrical pathways; and means for outputting the optimized electro-anatomical model.

Illustrative embodiment 11. The apparatus according to illustrative embodiment 10, wherein the means for optimizing the electro-anatomical model based on one or more electrical conduction parameters for electrical pathways in the electro-anatomical model comprises: means for optimizing the electro-anatomical model by activating a proportion of the electrical pathways in the electro-anatomical model determined based on the one or more electrical conduction parameters.

Illustrative embodiment 12. The apparatus according to any one of illustrative embodiments 10-11, wherein the means for optimizing the electro-anatomical model based on one or more electrical conduction parameters for electrical pathways in the electro-anatomical model comprises: means for optimizing the electro-anatomical model using conduction velocity parameters determined by multiplying a maximal conduction velocity of a fascicle by the one or more electrical conduction parameters.

Illustrative embodiment 13. The apparatus according to any one of illustrative embodiments 10-12, wherein the means for optimizing the electro-anatomical model based on one or more electrical conduction parameters for electrical pathways in the electro-anatomical model comprises: means for optimizing the electro-anatomical model by delaying electrical activation in the electrical pathways based on the one or more electrical conduction parameters.

Illustrative embodiment 14. The apparatus according to any one of illustrative embodiments 10-13, wherein the anatomical object comprises a heart of the patient, the value representing no electrical conduction in the electrical pathways corresponds to complete conduction block, and the value representing full electrical conduction in the electrical pathways corresponds to intact Purkinje activation.

Illustrative embodiment 15. A non-transitory computer-readable storage medium comprising instructions which, when executed by a computer, cause the computer to carry out operations comprising: receiving 1) one or more input medical images of an anatomical object of a patient and 2) electrophysiological data associated with the anatomical object; generating an electro-anatomical model of the anatomical object based on the one or more input medical images and the electrophysiological data; optimizing the electro-anatomical model based on one or more electrical conduction parameters for electrical pathways in the electro-anatomical model, wherein the one or more electrical conduction parameters are continuous variables defined between a value representing no electrical conduction in the electrical pathways and a value representing full electrical conduction in the electrical pathways; and outputting the optimized electro-anatomical model.

Illustrative embodiment 16. The non-transitory computer-readable storage medium according to illustrative embodiment 15, wherein optimizing the electro-anatomical model based on one or more electrical conduction parameters for electrical pathways in the electro-anatomical model comprises: optimizing the electro-anatomical model by activating a proportion of the electrical pathways in the electro-anatomical model determined based on the one or more electrical conduction parameters.

Illustrative embodiment 17. The non-transitory computer-readable storage medium according to any one of illustrative embodiments 15-16, the operations further comprising: simulating a medical procedure on the anatomical object using the optimized electro-anatomical model.

Illustrative embodiment 18. The non-transitory computer-readable storage medium according to illustrative embodiment 17, wherein the anatomical object is a heart of the patient and simulating a medical procedure on the anatomical object using the optimized electro-anatomical model comprises: simulating different types of cardiac resynchronization therapy for determining an optimal treatment for bundle branch block of the electrical pathways.

Illustrative embodiment 19. The non-transitory computer-readable storage medium according to illustrative embodiment 18, wherein the different types of cardiac resynchronization therapy comprise left ventricle pacing, bi-ventricular pacing, His bundle pacing, and Left bundle branch pacing (LBBP).

Illustrative embodiment 20. The non-transitory computer-readable storage medium according to any one of illustrative embodiments 15-19, wherein the anatomical object comprises a heart of the patient, the value representing no electrical conduction in the electrical pathways corresponds to complete conduction block, and the value representing full electrical conduction in the electrical pathways corresponds to intact Purkinje activation.

1. A computer-implemented method comprising:
receiving 1) one or more input medical images of an anatomical object of a patient and 2) electrophysiological data associated with the anatomical object;
generating an electro-anatomical model of the anatomical object based on the one or more input medical images and the electrophysiological data;
optimizing the electro-anatomical model based on one or more electrical conduction parameters for electrical pathways in the electro-anatomical model, wherein the one or more electrical conduction parameters are continuous variables defined between a value representing no electrical conduction in the electrical pathways and a value representing full electrical conduction in the electrical pathways; and
outputting the optimized electro-anatomical model.

2. The computer-implemented method of claim 1, wherein optimizing the electro-anatomical model based on one or more electrical conduction parameters for electrical pathways in the electro-anatomical model comprises:
optimizing the electro-anatomical model by activating a proportion of the electrical pathways in the electro-anatomical model determined based on the one or more electrical conduction parameters.

3. The computer-implemented method of claim 1, wherein optimizing the electro-anatomical model based on one or more electrical conduction parameters for electrical pathways in the electro-anatomical model comprises:
optimizing the electro-anatomical model using conduction velocity parameters determined by multiplying a maximal conduction velocity of a fascicle by the one or more electrical conduction parameters.

4. The computer-implemented method of claim 1, wherein optimizing the electro-anatomical model based on one or more electrical conduction parameters for electrical pathways in the electro-anatomical model comprises:
optimizing the electro-anatomical model by delaying electrical activation in the electrical pathways based on the one or more electrical conduction parameters.

5. The computer-implemented method of claim 1, further comprising:
simulating a medical procedure on the anatomical object using the optimized electro-anatomical model.

6. The computer-implemented method of claim 5, wherein the anatomical object is a heart of the patient and simulating a medical procedure on the anatomical object using the optimized electro-anatomical model comprises:
simulating different types of cardiac resynchronization therapy for determining an optimal treatment for bundle branch block of the electrical pathways.

7. The computer-implemented method of claim 6, wherein the different types of cardiac resynchronization therapy comprise left ventricle pacing, bi-ventricular pacing, His bundle pacing, and Left bundle branch pacing.

8. The computer-implemented method of claim 1, wherein the anatomical object comprises a heart of the patient, the value representing no electrical conduction in the electrical pathways corresponds to complete conduction block, and the value representing full electrical conduction in the electrical pathways corresponds to intact Purkinje activation.

9. The computer-implemented method of claim 1, wherein the anatomical object comprises a heart of the patient and the electrical pathways comprise at least one of a bundle of His, a right bundle branch, a left branch, Purkinje fibers, or any sub-branch thereof.

10. An apparatus comprising:
means for receiving 1) one or more input medical images of an anatomical object of a patient and 2) electrophysiological data associated with the anatomical object;
means for generating an electro-anatomical model of the anatomical object based on the one or more input medical images and the electrophysiological data;
means for optimizing the electro-anatomical model based on one or more electrical conduction parameters for electrical pathways in the electro-anatomical model, wherein the one or more electrical conduction parameters are continuous variables defined between a value representing no electrical conduction in the electrical pathways and a value representing full electrical conduction in the electrical pathways; and
means for outputting the optimized electro-anatomical model.

11. The apparatus of claim 10, wherein the means for optimizing the electro-anatomical model based on one or more electrical conduction parameters for electrical pathways in the electro-anatomical model comprises:
means for optimizing the electro-anatomical model by activating a proportion of the electrical pathways in the electro-anatomical model determined based on the one or more electrical conduction parameters.

12. The apparatus of claim 10, wherein the means for optimizing the electro-anatomical model based on one or more electrical conduction parameters for electrical pathways in the electro-anatomical model comprises:
means for optimizing the electro-anatomical model using conduction velocity parameters determined by multiplying a maximal conduction velocity of a fascicle by the one or more electrical conduction parameters.

13. The apparatus of claim 10, wherein the means for optimizing the electro-anatomical model based on one or more electrical conduction parameters for electrical pathways in the electro-anatomical model comprises:
means for optimizing the electro-anatomical model by delaying electrical activation in the electrical pathways based on the one or more electrical conduction parameters.

14. The apparatus of claim 10, wherein the anatomical object comprises a heart of the patient, the value representing no electrical conduction in the electrical pathways corresponds to complete conduction block, and the value representing full electrical conduction in the electrical pathways corresponds to intact Purkinje activation.

15. A non-transitory computer-readable storage medium comprising instructions which, when executed by a computer, cause the computer to carry out operations comprising:
receiving 1) one or more input medical images of an anatomical object of a patient and 2) electrophysiological data associated with the anatomical object;
generating an electro-anatomical model of the anatomical object based on the one or more input medical images and the electrophysiological data;
optimizing the electro-anatomical model based on one or more electrical conduction parameters for electrical pathways in the electro-anatomical model, wherein the one or more electrical conduction parameters are continuous variables defined between a value representing no electrical conduction in the electrical pathways and a value representing full electrical conduction in the electrical pathways; and
outputting the optimized electro-anatomical model.

16. The non-transitory computer-readable storage medium of claim 15, wherein optimizing the electro-anatomical model based on one or more electrical conduction parameters for electrical pathways in the electro-anatomical model comprises:
optimizing the electro-anatomical model by activating a proportion of the electrical pathways in the electro-anatomical model determined based on the one or more electrical conduction parameters.

17. The non-transitory computer-readable storage medium of claim 15, the operations further comprising:
simulating a medical procedure on the anatomical object using the optimized electro-anatomical model.

18. The non-transitory computer-readable storage medium of claim 17, wherein the anatomical object is a heart of the patient and simulating a medical procedure on the anatomical object using the optimized electro-anatomical model comprises:
simulating different types of cardiac resynchronization therapy for determining an optimal treatment for bundle branch block of the electrical pathways.

19. The non-transitory computer-readable storage medium of claim 18, wherein the different types of cardiac resynchronization therapy comprise left ventricle pacing, bi-ventricular pacing, His bundle pacing, and Left bundle branch pacing.

20. The non-transitory computer-readable storage medium of claim 15, wherein the anatomical object comprises a heart of the patient, the value representing no electrical conduction in the electrical pathways corresponds to complete conduction block, and the value representing full electrical conduction in the electrical pathways corresponds to intact Purkinje activation.
